(19) United States
(12) Patent Application Publication
(10) Pub. No.: US 2013/0217992 A1
(43) Pub. Date: Aug. 22, 2013

(54) Electrode Device, in Particular for Cardiovascular Application

(71) Applicant: BIOTRONIK SE & CO. KG, (US)

(72) Inventors: Klaus Bartels, Berlin (DE); Thomas Guenther, Michendorf (DE); Carsten Steglich, Berlin (DE)

(73) Assignee: BIOTRONIK SE & CO. KG, Berlin (DE)

(21) Appl. No.: 13/748,044

(22) Filed: Jan. 23, 2013

Related U.S. Application Data
(60) Provisional application No. 61/599,429, filed on Feb. 16, 2012.

(51) Int. Cl. A61B 5/042; A61N 1/362; A61N 1/05; A61B 5/00

(52) U.S. Cl. CPC A61B 5/0422; A61B 5/4836; A61N 1/362; A61N 1/05
USPC 600/374

(57) ABSTRACT

An electrode device, in particular for cardiovascular applications, includes an elongated electrode body made from an insulating material, a plurality of electrodes for detecting electrocardiological signals and/or for outputting electrocardiological stimulus signals, and supply line, in particular non-elastic cables or strands, which serve for electrically connecting the electrodes, which supply lines are guided in each case in the electrode body, preferably in associated lumina. Furthermore, a compensating hose section is provided which is inserted in a parting point in the electrode and has a maximum outer diameter that corresponds to the electrode body, wherein helically shaped receptacles, for each supply line, are incorporated in the compensating hose section, and the compensating hose section, at its joining sides facing toward the electrode body, is connected in a hermetically sealed manner to the electrode body.

### CROSS-REFERENCE TO RELATED APPLICATIONS

This patent application claims the benefit of co-pending U.S. Provisional Patent Application No. 61/599,429, filed on Feb. 16, 2012, which is hereby incorporated by reference in its entirety.

### TECHNICAL FIELD

The present invention generally relates to electrode devices and, in particular, to an electrode device for cardiovascular application with at least the features specified in claim 1.

### BACKGROUND

Such electrode devices as they are known, for example, from U.S. Pat. No. 7,238,883, U.S. Publication No. 2007/0225784 and/or U.S. Pat. No. 7,395,116, are known to have an elongated electrode body made from an insulating material, one or a plurality of electrodes for detecting cardiological signals and/or for outputting electrocardiological stimulus signals, as well as supply lines serving for the electrical connection of the electrodes. The supply lines are, in each case, guided in adequate lumina in the electrode body.

Such electrode devices which are used, for example, as pacemakers, defibrillators and other multi-electrode systems, are utilized for diagnostic and therapeutic purposes. As supply lines, more and more often, non-elastic electrical conductors are employed such as, for example, cables and strands.

Due to the eccentric arrangement of a plurality of conductors of this non-elastic type in multi-lumen constructions, as well as in lumenless arrangements, a relative movement between these conductors and the electrode insulation in a multi-lumen arrangement, or among the insulated supply lines in a lumenless construct, is generally unavoidable. Naturally, in the case of one or a plurality of cables which show little play in their guiding lumina of the insulating electrode body and which, furthermore, with regard to their cross-section, are arranged radially on the outside or asymmetrically about a lumen guiding a central coil conductor and, therefore, do not lie in the neutral phase of the electrode body, this, depending on the stress state, results in friction between the involved components which, in particular in the case of extensive bending movements within an electrode body, can be significant compared to a conventional coaxial structure consisting of, in each case, elastic inner and outer conductors in the form of coils.

In order to optimize the manufacturing process, electrode devices with a plurality of individual electrodes, as they are used for stimulating and sensing heart action potentials, for defibrillation or for connecting sensors or actuators, often consist of prefabricated subassemblies which have to be assembled during the course of the manufacture. In this connection, it is known for conventional electrode devices to establish such connections by means of adhesively bonding a transfer sleeve, which serves as a connection coupling and acts as a kind of “bandage”, around the joint between the prefabricated sub-components. For example, a shock electrode subassembly of a defibrillator electrode device can be assembled with the actual elongated electrode body in the aforementioned manner. The transitions with transfer sleeves can represent interference points in the form of sudden diameter changes in the otherwise isodiametric shape of the electrode body, which interference points, after an implantation of the electrode device, can be the cause of increased tissue growth which can affect the function of the system.

From the prior art, it is known through prior public use to solve the problem of non-elastic cables and strands as a conductor by twisting the cables. However, this measure is known only directly at the connection of the cable or the strand to a plug connector and serves there exclusively for increasing the bending fatigue strength at the transition between conductor cable and plug connector.

Another known solution is the transfer of the conductor cable into an elastic coil. The problem here is the fact that by this transition between a cable and a wire coil in the high voltage path of an electrode device, the electrical properties of the electrode device are significantly diminished. In this respect, this prior art is not feasible.

The present invention is directed toward overcoming one or more of the above-identified problems.

Proceeding from the above-described problems of the prior art, it is an object of the present invention to refine an electrode device in such a manner that movements of the supply lines, in particular if they are configured as non-elastic cables or strands, relative to the insulating electrode body are minimized, and that for the implementation, connection couplings or transfer sleeves are avoided by suitably designing the electrode device.

### SUMMARY

At least the above object is achieved by the features disclosed in the independent claim(s). According to that, a compensating hose section inserted in a parting point in the electrode body is provided, wherein the compensating hose section has a maximum diameter that corresponds to the electrode body. Helically shaped receptacles for each supply line are incorporated in this compensating hose section. Furthermore, with its joining sides facing toward the electrode body, the compensating hose section is connected to the latter in a hermetically sealed manner.

By laying the supply lines in a helically shaped configuration in the region of the compensating hose section, the electrode body can be subjected to bending influences in the region of said compensating hose section without any problems because tensile loads acting on the line section situated on the outer side of the bend are compensated by the compaction of this supply line on the inner side of the bend within the compensating hose section. In this respect, the electrode body is therefore particularly flexible in the region of this compensating hose section without significant internal friction forces being generated. Advantageously, due to the increased flexibility in the region of the compensating hose sections, such electrode devices are physiologically more compatible. With regard to product safety, electrode devices with multi-lumen structures become more reliable because they are less sensitive to bending load alternations. This is also facilitated by the reduced friction between the conductors in the adjacent lumen in the case of a multi-lumen structure, or between adjacent insulated conductors in the case of a lumenless construction.

By connecting the compensating hose sections to the adjacent electrode body via a hermetically sealed connection at their joining sides toward the electrode body, furthermore, transfer sleeves and the corresponding work steps to attach the same can be eliminated. Also, with the sleeveless construction, sudden diameter changes are avoided and an isodiametric structure of the electrode device is ensured.

According to a preferred embodiment of the electrode device, the receptacles for the supply lines in the compensating hose section can be formed as helically shaped lumina or as helically shaped grooves which are open on the outer side.

In the latter case, the compensating hose section with its grooves receiving the supply lines is preferably enclosed on the outside with a cover sleeve. In contrast to the prior art, however, this is unproblematic with regard to an isodiametric structure of the electrode device because, in this case, the compensating hose section has a radius that is reduced by the wall thickness of the cover sleeve.

A particularly preferred embodiment of the present invention is obtained when using an electrode device which comprises at least one shock coil on the electrode body. The shock coil is usually formed from a conductive helically wound coil wire. Particularly preferred in this case is the arrangement of the compensating hose section at least partially underneath this shock coil, wherein at least one joining side of the compensating hose section that faces toward the electrode body lies underneath the shock coil. Via a step with a reduced diameter, the electrode coil then engages in the shock coil up to the joining side of the compensating hose section.

Thereby, the parting point between the electrode body and the compensating hose section is placed in an advantageous manner underneath the shock coil, which is beneficial for forming an ideally homogenous, isodiametric electrode device. The diameter-reduced steps of the electrode body can be machined, fir example, by milling, but also by grinding, laser ablation, and the like.

The transfer point between the compensating hose section and the electrode body is particularly protected if the gaps between the hose section and the shock coil are filled, for example, with a grouting agent.

If the electrode device comprises a centrally guided, elastic supply line and, in particular, a coiled line, it is provided as a further preferred embodiment that the compensating hose section includes a central, straight and continuous lumen for this elastic supply line. Thus, the compensating hose section can be widely used for a multiplicity of different supply line configurations.

Further preferred embodiments relate to the material selection and corresponding provision of the compensating hose section which can consist of, for example, silicone rubber. Manufacturing can be carried out by, for example, extrusion or injection molding of liquid silicone rubber.

When using a cover sleeve for the parting point, it is advantageous in terms of the material to produce the compensating hose section from silicone rubber or silicone polyurethane copolymer and to produce the cover sleeve from the last mentioned material or from polyurethane. The last mentioned materials show a good abrasion resistance which is in particular relevant for the outer cover sleeve. Of course, other materials having similar properties are contemplated.

Finally, a pitch of the helically shaped receptacles in the compensating hose section which corresponds to the 3- to 5-fold of the outer diameter of the compensating hose section has been found to be advantageous with regard to a compensation of the bending loads and a corresponding reduction of the internal friction. Thereby, the extent of the elongation of the supply lines in the region of the compensating section is limited to an acceptable level.

Further features, aspects, objects, advantages, and possible applications of the present invention will become apparent from a study of the exemplary embodiments and examples described below, in combination with the figures, and the appended claims.

### DESCRIPTION OF THE DRAWINGS

Further features, details and advantages of the present invention arise from the following description of exemplary embodiments based on the attached drawings. In the figures:

FIG. 1 shows a sectional, partially cut out side view of an electrode device of the present invention in a first embodiment;

FIG. 2 shows a view of the electrode device according to FIG. 1 in an exploded view;

FIG. 3 shows a side view of the electrode device in a second embodiment; and

FIG. 4 shows a view of the electrode device according to FIG. 3 in an exploded view.

### DETAILED DESCRIPTION

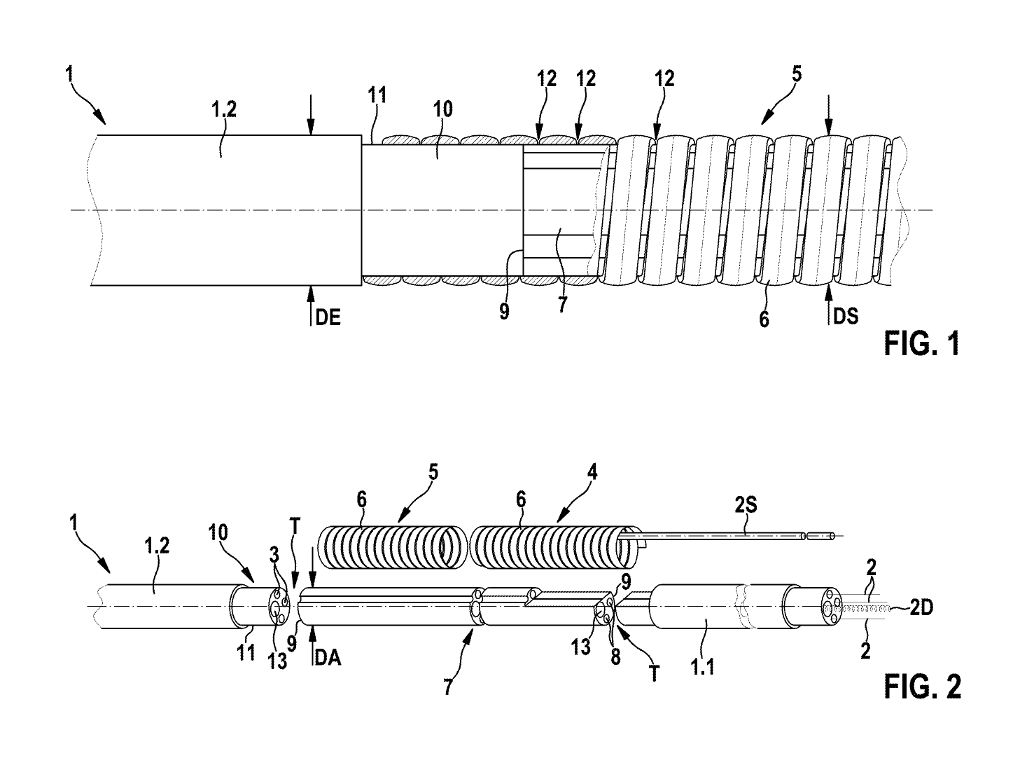

As is apparent from FIGS. 1 and 2, the partially shown electrode device for cardiovascular applications has an elongated electrode body 1 which is separated at a parting point T and, thus, is divided into two segments 1.1, 1.2. The electrode body 1 is an elongated, hose-like construct made from an insulating material in which a plurality of supply lines 2 to the corresponding electrodes for detecting cardiological signals and/or for outputting electrocardiological stimulus signals are guided in lumina 3. FIGS. 1 and 2 show two shock coils 4, 5 of these electrodes, the shock coils 4, 5 being formed from a tightly wound tape wire 6. The outer diameter DS of the shock coils 4, 5 corresponds substantially to the outer diameter DE of the electrode body 1 in the two sections 1.1, 1.2 of the latter. Apart from that, FIG. 2 shows the supply line 2S of the shock coil 4.

The other supply lines 2, which are only indicated in FIG. 2, are configured as non-elastic cables.

In the parting point T between the two sections 1.1, 1.2 of the electrode body 1, a compensating hose section 7 is inserted which has receptacles 8 in the form of lumina corresponding to the lumina 3. As not explicitly illustrated in FIGS. 1 and 2, these receptacles 8 are formed helically along the longitudinal direction of the compensating hose section 7 so that the supply lines 2 guided therein also run in a helically shaped manner. The outer diameter DA of the compensating hose section 7 is adapted to the inner diameter of the shock coil 4, 5 so that the compensating hose section 7 is arranged within the shock coil 4, 5. The transversely extending joining sides 9 of the hose section 7 are retracted inwardly with respect to the outer ends 10 of the shock coils 4, 5. For connecting to the sections 1.1, 1.2 of the electrode body 1, said sections have diameter-reduced steps 11, the outer diameters of which correspond to the diameter DA of the compensating hose sections 7. Therewith, the steps 11 engage with the shock coils 4, 5 up the joining side 9 of the hose section 7. The latter is then, in each case, hermetically glued in a solution-tight manner to the sections 1.1, 1.2 of the electrode body 1. Furthermore, the intermediate spaces 12 between the shock coils 4, 5 and the compensating hose section 7 are filled with a grouting agent, which is not illustrated in detail, for example, in the form of liquid silicone rubber or silicone adhesive. Since the compensating hose section 7 itself is extruded from silicone rubber or is produced from liquid silicone rubber by means of injection molding, the silicone materials of the hose section 7 and the grouting agent bond in a non-detachable manner.

Apart from that, FIG. 2 also shows a central, straight and continuous lumen 13 for a helically shaped elastic supply line 2D of a tip electrode, which is not shown in detail. For clarity reasons, the supply lines 2, 2S and 2D are illustrated coming in only from the right side before the section 1.2 of the electrode body 1.

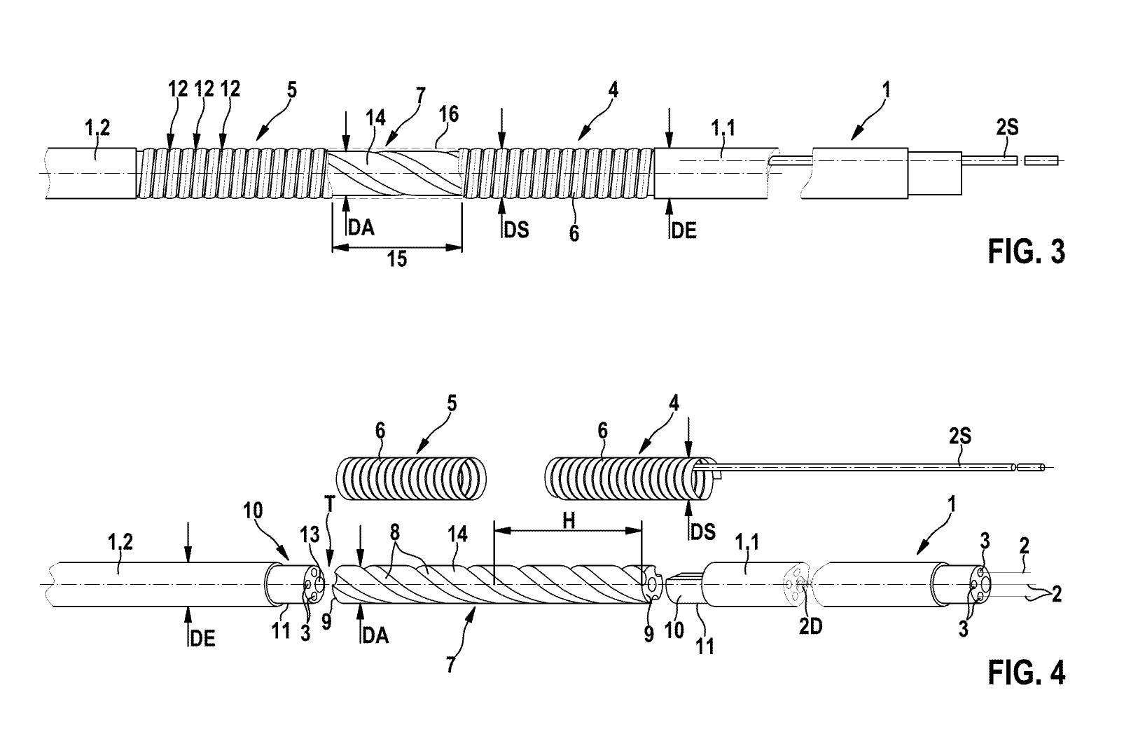

FIGS. 3 and 4 show again the cut-out of an electrode body 1 in which between the two sections 1.1 and 1.2, a compensating hose section 7 is inserted. In this embodiment, the latter comprises receptacles 8 which are formed as helical grooves on the outer side 14 thereof. Said grooves correspond with the three narrow lumina 3 in the electrode body 1 and receive the supply lines 2, 2S and 2D extending there through, wherein the supply lines are indicated again only on the right side in FIG. 4. A central lumen 13 receives a coil-shaped elastic supply line 2D. Analogous to the embodiment according to FIGS. 1 and 2, the compensating hose section 7 is arranged with its joining sides 9 underneath the two shock coils 4 and 5 and likewise connected in a hermetically sealed manner to the sections 1.1, 1.2 of the electrode body 1. The engagement of these sections 1.1, 1.2 in the shock coils 4, 5 is ensured again via the steps 11.

Between the two shock coils 4, 5 remains an intermediate space 15 in which a cover sleeve 16, illustrated with a dashed line in FIG. 3, is placed onto the compensating hose section 7 having the groove-like receptacles 8 and is adhered thereto in a hermetically sealed manner. The outer diameter DA of this cover sleeve 16 corresponds to the outer diameter DS of the shock coils 4, 5, so that overall an isodiametric structure of the electrode device according to FIGS. 3 and 4 is obtained. This applies also to the embodiment shown in FIGS. 1 and 2.

Corresponding to the three lumina 3 in the electrode body 1, the compensating hose section 7 also has three groove-shaped receptacles 8 which each have a pitch H that corresponds approximately to the 4-fold of the outer diameter DA of the compensating hose section 7 in FIGS. 3 and 4.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An electrode device for cardiovascular applications, comprising:
an elongated electrode body made from an insulating material;
a plurality of electrodes for detecting electrocardiological signals and/or for out-putting electrocardiological stimulus signals;
supply lines, which include non-elastic cables or strands, serving for electrically connecting the electrodes, wherein the supply lines are in each case guided in the electrode body in associated lumina; and
a compensating hose section which is inserted in a parting point in the electrode body and has a maximum outer diameter that corresponds to the electrode body, wherein helically shaped receptacles for each supply line are incorporated in the compensating hose section, and the compensating hose section, at its joining sides facing toward the electrode body, is connected in a hermetically sealed manner to the electrode body.

2. The electrode device according to claim 1, wherein the receptacles are configured as helically shaped lumina.

3. The electrode device according to claim 1, wherein the receptacles are configured as helically shaped grooves on an outer side of the compensating hose section.

4. The electrode device according to claim 3, wherein the compensating hose section, with its groove-shaped receptacles receiving the supply lines, is enclosed on the outside with a cover sleeve.

5. The electrode device according to claim 1, further comprising at least one shock coil on the electrode body, the shock coil being formed from a conductive, helically wound coil wire, wherein the compensating hose section is arranged at least partially underneath the at least one shock coil, wherein at least one joining side of the compensating hose section facing toward the electrode body lies underneath the at least one shock coil, and the electrode body, with a diameter-reduced step, engages in the at least one shock coil up to the joining side of the compensating hose section.

6. The electrode device according to claim 5, wherein intermediate spaces between the compensating hose section and the at least one shock coil are filled with a grouting agent.

7. The electrode device according to claim 1, further comprising a centrally guided elastic supply line, wherein the compensating hose section has a central, straight and continuous lumen for the elastic supply line.

8. The electrode device according to claim 1, wherein the elastic supply line comprises a coiled line.

9. The electrode device according to claim 1, wherein the compensating hose section is made of silicone rubber.

10. The electrode device according to claim 9, wherein the compensating hose section is extruded.

11. The electrode device according to claim 9, wherein the compensating hose section is injection molded from liquid silicone rubber.

12. The electrode device according to claim 4, wherein the compensating hose section is made of silicone rubber and/or silicone polyurethane copolymer, and the cover sleeve is made of silicone polyurethane copolymer and/or polyurethane.

13. The electrode device according to claim 1, wherein the pitch of a helically shaped receptacle corresponds to a 3-fold to 5-fold outer diameter of the compensating hose section.
